(19) United States
(12) Patent Application Publication
(10) Pub. No.: US 2024/0368657 A1
(43) Pub. Date: Nov. 7, 2024

(54) INCLUSION BODY MEDIATED METHOD FOR DOUBLE-STRANDED RNA PRODUCING

(71) Applicant: XIAOMO BIOTECH LIMITED, HONG KONG (CN)

(72) Inventors: LINFENG HUANG, HONG KONG (CN); PANPAN HUANG, HONG KONG (CN)

(21) Appl. No.: 18/748,282

(22) Filed: Jun. 20, 2024

Related U.S. Application Data
(63) Continuation of application No. PCT/CN2023/116412, filed on Sep. 1, 2023.

(30) Foreign Application Priority Data
Sep. 2, 2022 (WO) PCT/CN2022/116876

(51) Int. Cl. C12P 19/34; C07K 14/005; C12N 9/22; C12N 15/113; C12N 15/70

(52) U.S. Cl. CPC C12P 19/34; C07K 14/005; C12N 9/22; C12N 15/113; C12N 15/70; C12Y 301/26003; C12N 2310/14; C12N 2770/20022; C12N 2770/38022

(57) ABSTRACT

An inclusion body mediated method for double-stranded RNA producing. The method includes: transforming the vector to bacteria; culturing and inducing the bacteria expressed an inclusion body; and extracting the inclusion body by the sonication or high-pressure lysis of the collected bacteria, wherein the vector at least includes a nucleic acid sequence encoding a siRNA-binding polypeptide that corporate p19 protein with protein 1.

### TECHNICAL FIELD

The technology described herein relates to methods for producing double-stranded RNA in bacterial cells.

### SEQUENCE LISTING

The instant application contains a Sequence Listing which has been submitted electronically in ST.26 format and is hereby incorporated by reference in its entirety. Said ST.26 copy, created on Jun. 11, 2024, is named SEQUENCE LISTING.xml and is 12,512 bytes in size.

### BACKGROUND

RNA interference (RNAi) by double-stranded (ds) small interfering RNAs (siRNA) suppresses gene expression by inducing the degradation of mRNAs bearing complementary sequences. Transfection of synthetic siRNAs into eukaryotic cells to silence genes has become an indispensable tool to investigate gene function, and siRNA-based therapy is being developed to knockdown genes implicated in disease.

A novel siRNA design and production system has been invented by Dr. Linfeng Huang (Huang et al, Nat Biotechnology, 2013, 31(4):350-6; Huang & Lieberman, Nat Protocol, 2013, 8(12):2325-36). This system utilizes the unique function of the protein p19, which can bind to and stabilize 21-nt double stranded RNA species produced by endogenous RNase III in Escherichia coli (E. coli), producing a pool of siRNAs within a certain selected gene sequence. Those siRNAs produced in E. coli are double-stranded RNA fragments.

However, there are still needs for more efficient ways to produce double-stranded RNAs.

### SUMMARY

The technology described herein is directed to methods, insoluble complex and vector relating to the production of double-stranded RNA generated in vivo, e.g. in bacterial cells.

In an aspect, a vector includes a first nucleic acid sequence encoding a fusion protein which is siRNA-binding polypeptide comprising a p19 protein and a protein named as protein 1 (P1) and a second nucleic acid sequence substantially complementary to a target RNA.

In an embodiment, the vector further comprises a third nucleic acid sequence encoding an siRNA-generating enzyme, and the siRNA-generating enzyme is selected from RNase III and/or RNase III mutant at least 95% identical to the RNase III.

In an embodiment, the p19 protein is selected from following proteins: Tombusvirus (TBSV) p19 protein; TBSV p19 protein mutant at least 95% identical to the TBSV p19 protein; Carnation Italian ringspot virus (CIRV) p19 protein; or CIRV p19 protein mutant at least 95% identical to the CIRV p19 protein.

In an embodiment, wherein the nucleic acid sequence encodes for protein 1 as set forth in SEQ ID NO: 1, SEQ ID NO: 2, SEQ ID NO: 3, SEQ ID NO: 4 or SEQ ID NO:5.

In an embodiment, the siRNA-binding polypeptide comprises a purification tag.

In an embodiment, the vector comprises a promoter sequence.

In an embodiment, a composition for targeting a disease further comprises one or more pharmaceutical carriers, excipients, preservatives, colorants and/or diluents.

In an embodiment, the vector is a plasmid expressed by E. coli cell.

In an aspect, an inclusion body expressed by bacteria, wherein the inclusion body encodes by the vector disclosed herein.

In an aspect, an insoluble complex is generated by the interaction of a fusion protein of p19 and a double stranded RNA of target sequence.

In an embodiment, the fusion protein of p19 at least comprises p19 protein and P1 protein.

In an aspect, a method for double-stranded RNA producing comprises: transforming the vector disclosed herein to bacteria; culturing and inducing the bacteria expressed the inclusion body; extracting the inclusion body by the sonication or high-pressure lysis of the collected bacteria; resuspending the inclusion body to produce the released double-stranded RNA.

In an embodiment, the bacterium is an E. coli cell.

In an embodiment, the method further comprises purifying the released double-stranded RNA.

In an embodiment, the step of extracting the inclusion body further comprises: crushing the collected bacteria by sonication or high-pressure lysis method and extracting the inclusion body from the crushed bacteria by centrifugation.

In an aspect, a double-stranded RNA produced by the method described as below.

In an aspect, a method of RNAi comprising application of the double-stranded RNA produced by the method described as below to a subject in need of RNA interference, wherein the subject comprises eukaryotic cells and/or organisms.

One advance of the method for double-stranded RNA producing disclosed by the embodiment is that the method incorporates p19 protein with a new aggregation protein domain named “protein 1” to form an insoluble form known as the “inclusion body”, such that the yield and length diversity of the inclusion-body formulated double-stranded RNA was increased greatly compared with traditional double-stranded RNA producing methods.

Another advance of the method for double-stranded RNA producing disclosed by the embodiments are that protein 19 and protein 1 also empower those double-stranded RNA with potentially built-in delivery efficacy.

### BRIEF DESCRIPTION OF THE DRAWINGS

In order to more clearly illustrate the technical solutions of the embodiments of the present application, a brief description will be given below of the accompanying drawings which are required to be used in the embodiments. It is to be understood that the following drawings illustrate only some embodiments of the present application and are therefore not to be considered as limiting the scope. For ordinary technical personnel in this field, other relevant drawings can also be obtained according to these drawings without involving any inventive effort.

FIG. 1 is a schematic diagram of the plasmid according to one embodiment of the present application.

FIG. 2 is a schematic diagram of the plasmid according to another embodiment of the present application, illustrates available options for the p19 protein, protein 1 and siRNA-generating enzyme.

FIG. 3A is a protein electrophoresis diagram of expression of P19-P1 fused protein according to embodiments of the present application.

FIG. 3B is a protein electrophoresis diagram of P19-P1 fused protein according to embodiments of the present application, illustrated different p19 protein species.

FIG. 3C is an electrophoresis diagram of double-stranded RNA produced by different P19-P1 fused proteins according to embodiments of the present application.

FIG. 3D is a protein electrophoresis diagram of P19-P1 fused protein with RNase III according to embodiments of the present application, illustrated different p19 protein species and RNase III mutants.

FIG. 3E is an electrophoresis diagram of double-stranded RNA produced by the P19-P1 fused protein according to FIG. 3D.

FIG. 3F is a protein electrophoresis diagram of purification of P19-P1 fused protein according to embodiments of the present application.

FIG. 4 is a flow chart of the method for double-stranded RNA producing according to one embodiment of the present application.

FIG. 5A is a schematic diagram illustrating the double-stranded RNA yield of a traditional method and an inclusion body method according to embodiments of the present application.

FIG. 5B is a schematic diagram illustrating the crude double-stranded RNA extracted by traditional method and inclusion body method according to embodiments of the present application before HPLC purification.

### DETAILED DESCRIPTION

In general, the terms and phrases used herein have their art-recognized meaning, which can be found by reference to standard texts, journal references and contexts known to those skilled in the art. The following definitions are provided to clarify their specific use in the context of this description.

Embodiments of the invention described herein are directed to methods, insoluble complex and vector relating to the production of double-stranded RNA in vivo, e.g. in bacterial cells (siRNAs produced according to the methods and compositions described herein are also referred to herein as “double-stranded RNA”). The technology described herein is derived from the inventors' discovery that the fusion of p19 protein and protein 1 forms an insoluble form known as the “inclusion body”. The yield and length diversity of the inclusion-body formulated double-stranded RNA was increased greatly compared with traditional double-stranded RNA producing methods. Beyond that, p19 protein and protein 1 also empower those double-stranded RNA (dsRNA) with potentially built-in delivery efficacy.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, the terms “reduced”, “reduction”, “decrease”, or “inhibit” can mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or more or any decrease of at least 10% as compared to a reference level. In some embodiments, the terms can represent a 100% decrease, i.e., a non-detectable level as compared to a reference level. In the context of a marker or symptom is meant a statistically significant decrease in such level. The decrease can be, for example, at least 10%, at least 20%, at least 30%, at least 40% or more, and is preferably down to a level accepted as within the range of normal for an individual without such disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “proteins” and “polypeptides” are used interchangeably herein to designate a series of amino acid residues connected to the other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA or cDNA. Other suitable nucleic acid molecules are RNA, including mRNA.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. A gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The term operatively linked includes having an appropriate start signal (e.g., ATG) in front of the polynucleotide sequence to be expressed and maintaining the correct reading frame to permit expression of the polynucleotide sequence under the control of the expression control sequence, and, optionally, production of the desired polypeptide encoded by the polynucleotide sequence. In some examples, transcription of a nucleic acid modulatory compound is under the control of a promoter sequence (or other transcriptional regulatory sequence) which controls the expression of the nucleic acid in a cell-type in which expression is intended. It will also be understood that the modulatory nucleic acid can be under the control of transcriptional regulatory sequences which are the same or which are different from those sequences which control transcription of the naturally occurring form of a protein.

The term “isolated” or “purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.”.

The term “complementary” or “complementary base pair” refers to A: T and G:C in DNA and A:U in RNA. Most DNA consists of sequences of nucleotide only four nitrogenous bases: base or base adenine (A), thymine (T), guanine (G), and cytosine (C). Together these bases form the genetic alphabet, and long ordered sequences of them contain, in coded form, much of the information present in genes. Most RNA also consists of sequences of only four bases. However, in RNA, thymine is replaced by uridine (U).

Other than in the operating examples, or where otherwise indicated, all numbers expressing quantities of ingredients or reaction conditions used herein should be understood as modified in all instances by the term “about.” The term “about” when used in connection with percentages can mean±1%.

The term “includes” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

The term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9). Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: A Comprehensive Desk Reference, published by VCH Publishers, Inc., 1995 (ISBN 1-56081-569-8) and Current Protocols in protein Sciences 2009, Wiley Intersciences, Coligan et al., eds.

Unless otherwise stated, the present invention can be performed using standard procedures, as described, for example in Sambrook et al., Molecular Cloning: A Laboratory Manual (3 ed.), Cold Spring Harbor Laboratory Press, Cold Spring Harbor, N.Y, USA (2001); Davis et al., Basic Methods in Molecular Biology, Elsevier Science Publishing, Inc., New York, USA (1995); Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

In the implementation of this application, applicant surprisingly find that a fusion protein of p19 and a double stranded RNAs of target sequence can interact and become an insoluble complex (i.e., inclusion body). Accordingly, the insoluble complex may use for purification of RNA product or their related application.

Specifically, the fusion protein of p19 may include p19 protein and protein 1. Alternatively, the p19 protein can be replaced by any suitable RNA-binding protein.

At least one advantage of the insoluble complex is that the RNAs of target sequence can be isolated from the protein and the cells according to the actual needs.

Furthermore, the bacteria cell lysates, insoluble particles (i.e., inclusion body) and the purified RNAs can be used for downstream applications.

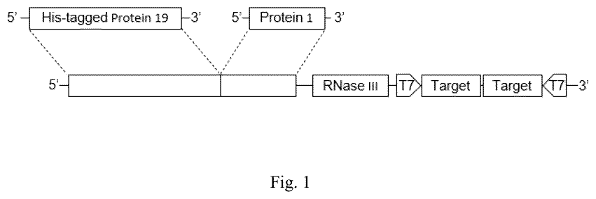

Referring to FIG. 1 illustrated the schematic of the vector which is used to express the inclusion body as described below. The vector includes a first nucleic acid sequence encoding a siRNA-binding polypeptide and a second nucleic acid sequence substantially complementary to a target RNA. The siRNA-binding polypeptide incorporate p19 protein with protein 1.

The term “vector”, as used herein, refers to a nucleic acid construct designed for delivery to a host cell or transfer between different host cells. As used herein, a vector can be viral or non-viral. Many vectors useful for transferring exogenous genes into target cells are available, e.g. the vectors may be episomal, e.g., plasmids, virus derived vectors or may be integrated into the target cell genome, through homologous recombination or random integration. In some embodiments, a vector can be an expression vector. As used herein, the term “expression vector” refers to a vector that has the ability to incorporate and express heterologous nucleic acid fragments in a cell. An expression vector may comprise additional elements, for example, the expression vector may have two replication systems, thus allowing it to be maintained in two organisms. The nucleic acid incorporated into the vector can be operatively linked to an expression control sequence when the expression control sequence controls and regulates the transcription and translation of that polynucleotide sequence. In some embodiments, the dsRNA and the nucleic acid encoding the siRNA-binding polypeptide can be within the same operon. In some embodiments, the dsRNA and the nucleic acid encoding the siRNA-binding polypeptide can be within separate operons.

As used herein, “p19 protein” can be also named as p19 polypeptides. “p19 protein” in particular but not limited to the p19 polypeptide such as tombusvirus p19 (NCBI Gene ID: 1493957) are able to bind to the siRNA and in particular include those as disclosed in US 2015/0337306A1 which are incorporated herein by reference. The p19 polypeptide as disclosed in US 2015/0337306A1, i.e. selected from a p19 polypeptide such as tombusvirus p19 polypeptide.

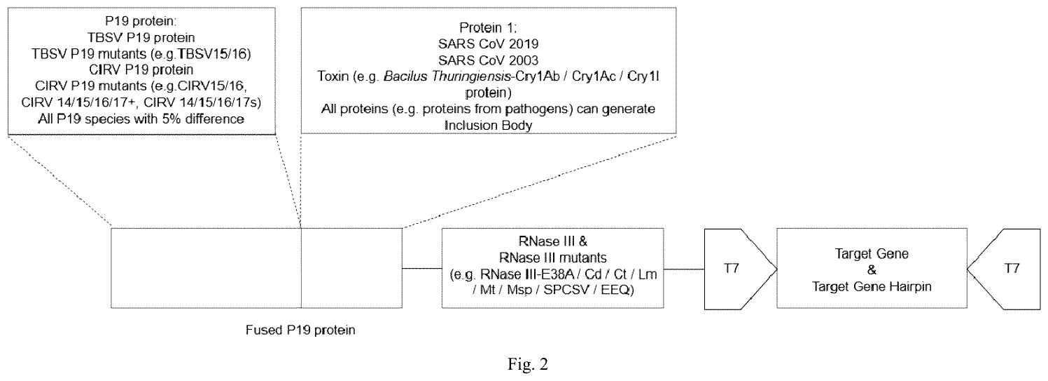

The term “protein 19” refers to a viral protein which binds specifically to dsRNAs and which suppresses RNAi-mediated host plant viral defenses. The sequences of p19 polypeptides from a number of species are known, e.g. tombusvirus p19 (NCBI Gene ID: 1493957). In some embodiments, referring to FIG. 2, the p19 polypeptide can be tombusvirus p19. Non-limiting examples of p19 homologues include Carnation Italian ringspot virus p19; Tomato bushy stunt virus p19; Artichoke mottled crinkle virus p19; Lisianthus necrosis virus p19; Pear latent virus p19; Cucumber Bulgarian virus p19; Cucumber necrosis virus p19; Pelargonium necrotic spot virus p19; Cymbidium ringspot virus p19; Lisianthus necrosis virus p19; Lettuce necrotic stunt virus p19; Maize necrotic streak virus p19; Grapevine Algerian necrosis virus p19; and Grapevine Algerian latent virus p19. A p19 polypeptide can comprise mutants, variants, homologues, and functional fragments of wildtype p19 polypeptides.

Further preferred, the siRNA-binding polypeptide has a purification tag suitable for purification of the siRNA-binding polypeptide and siRNAs bound to the siRNA-binding polypeptide. The purification tag can bind to another moiety such as on a matrix or a resin with affinity for the purification tag such as Ni-NTA resin. Particular purification tags include histidine tags (“His-tagged”) such as disclosed in US 2015/0337306A1 which are incorporated herein by reference. The siRNA-binding polypeptide is in particular a His-tagged p19 polypeptide.

The vector is in particular able to express a siRNA-binding polypeptide, in particular a p19 polypeptide such as His-tagged p19 polypeptide, and an siRNA-generating enzyme like a RNase III or RNase III mutants, in particular p19 such as His-tagged p19, fused to a siRNA-generating enzyme like an E. coli RNase III. Such expression of siRNA-generating enzyme like an E. coli RNase III will enhance the siRNA production.

As used herein, “protein 1” refers to a new aggregation protein domain. The fusion of p19 protein and protein 1 forms inclusion body to achieve significant yield improvement of the double-stranded RNAs.

In some embodiments, referring to FIG. 2, the protein 1 can be Severe acute respiratory syndrome coronavirus (SARS CoV) 2019, SARS CoV 2003, toxin; or any suitable protein configured to generate inclusion body, as set forth in SEQ ID NO:1-5. Specifically, SEQ ID NO:1 illustrates fusion protein that is p19 protein+protein 1 that is SARS CoV 2019, SEQ ID NO:2 illustrates protein 1 that is SARS CoV 2003, SEQ ID NO:3 illustrates protein 1 that is Bacillus thuringiensis-Cry1Ab, SEQ ID NO:4 illustrates protein 1 that is Bacillus thuringiensis-Cry1Ac, and SEQ ID NO:4 illustrates protein 1 that is Bacillus thuringiensis-Cry1I.

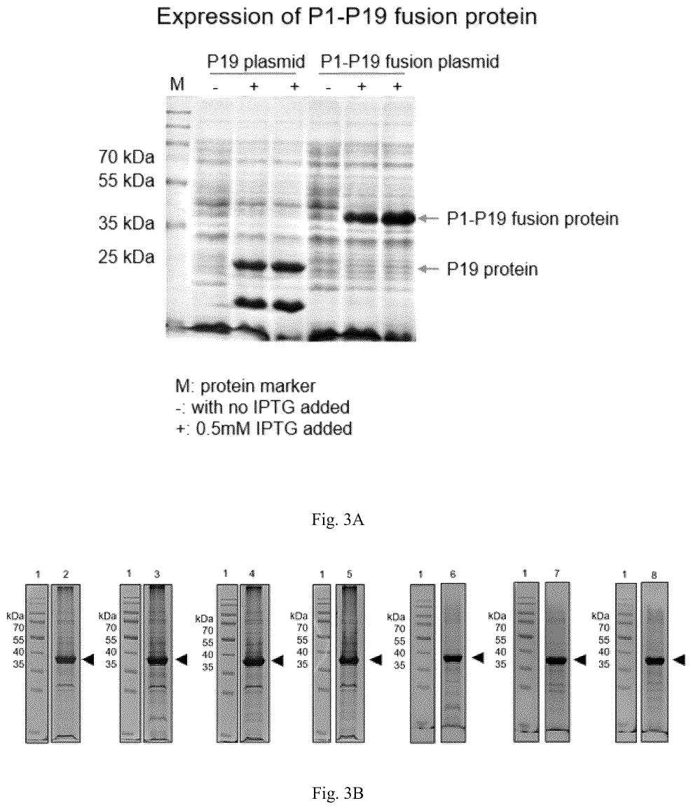

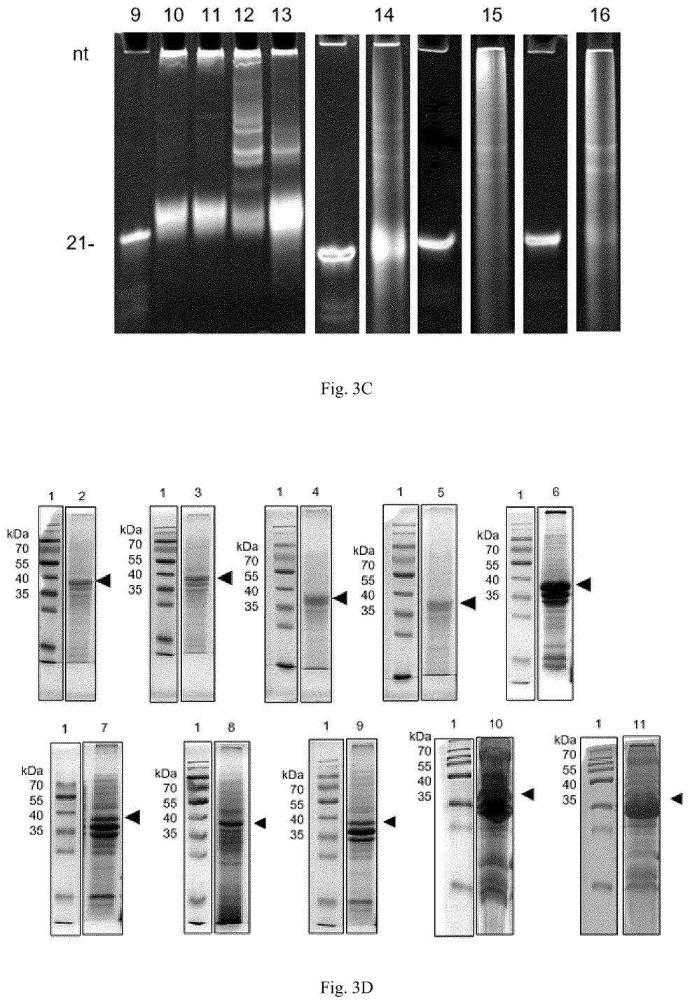

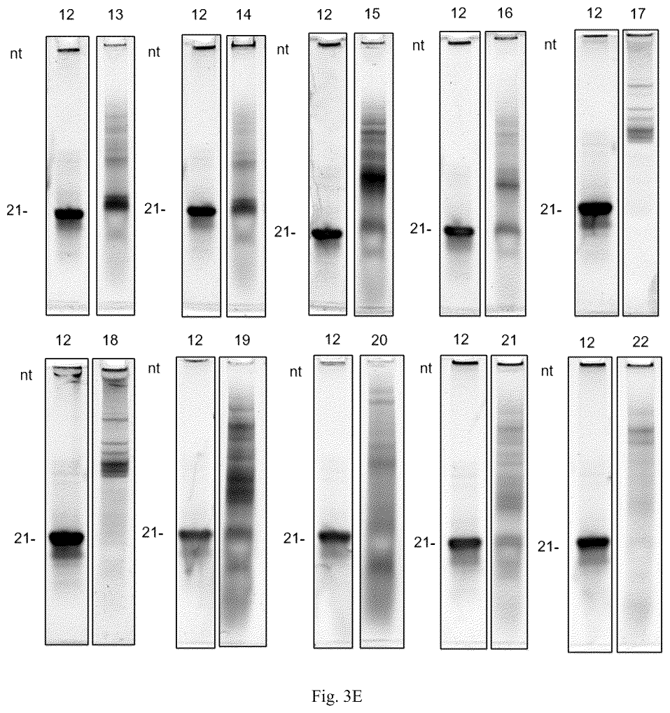

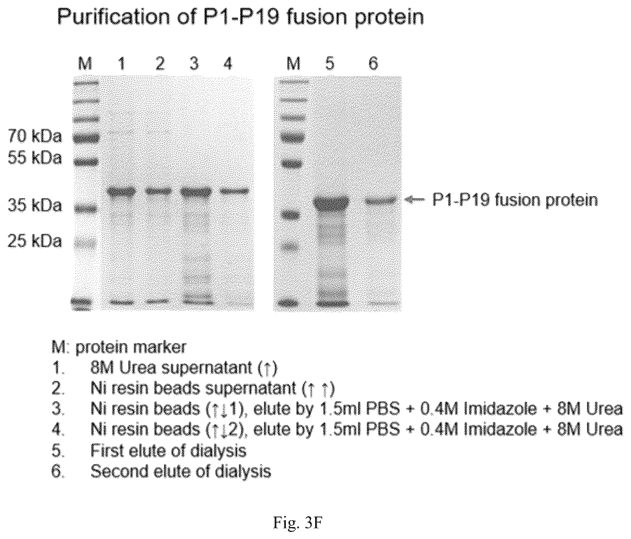

The parameters of fusion protein and their dsRNA products can be shown in FIG. 3A-3F. FIGS. 2B-2D illustrate inclusion body and their dsRNA products generated by different composition of p19 protein, p19 mutant, P1, RNase III and RNase III mutant.

As shown in FIG. 3B, the first lane is protein ladder, the second lane is original p19-SARS CoV 2019+RNase III generated inclusion body, the third lane is TBSV p19-SARS CoV 2019+RNase III generated inclusion body, the fourth lane is CIRV p19-SARS CoV 2019+RNase III generated inclusion body, the fifth lane is CIRV 15/16 p19 mutant-SARS CoV 2019+RNase III generated inclusion body, the sixth lane is TBSV 15/16 p19 mutant-SARS CoV 2019+RNase III generated inclusion body, the seventh lane is CIRV 14/15/16/17+p19 mutant-SARS CoV 2019+RNase III generated inclusion body, the eighth lane is CIRV 14/15/16/17s p19 Mutant-SARS CoV 2019+RNase III generated inclusion body.

As shown in FIG. 3C, the ninth lane is chemically synthesized siRNA, the tenth lane is original p19-SARS CoV 2019+RNase III produced dsRNA, the eleventh lane is TBSV p19-SARS CoV 2019+RNase III produced dsRNA, the twelfth lane is CIRV p19-SARS CoV 2019+RNase III produced dsRNA, the 13^{th} lane is CIRV 15/16 p19 mutant-SARS CoV 2019+RNase III produced dsRNA, the 14^{th} lane is TBSV 15/16 p19 mutant-SARS CoV 2019+RNase III produced dsRNA, the 15^{th} lane is CIRV 14/15/16/17+p19 mutant-SARS CoV 2019+RNase III produced dsRNA, the 16^{th} lane is CIRV 14/15/16/17s p19 mutant-SARS CoV 2019+RNase III produced dsRNA.

As shown in FIG. 3D, the first lane is protein ladder, the second lane is CIRV 15/16 p19 mutant-SARS CoV 2019+RNase III C. trachomatis (Ct) generated inclusion body, the third lane is CIRV 15/16 p19 mutant-SARS CoV 2019+RNase III L. monocytogenes (Lm) generated inclusion body, the fourth lane is CIRV 15/16 p19 mutant-SARS CoV 2019+RNase III M. tuberculosis (Mt) generated inclusion body, the fifth lane is CIRV 15/16 p19 mutant-SARS CoV 2019+RNase III Mycoplasma sp. (Msp) generated inclusion body, the sixth lane is TBSV p19-SARS CoV 2019+RNase III E38A mutant generated inclusion body, the seventh lane is CIRV 15/16 p19 mutant-SARS CoV 2019+RNase III E38A mutant generated inclusion body, the eighth lane is TBSV p19-SARS CoV 2019+RNase III EEQ mutant generated inclusion body, the ninth lane is CIRV 15/16 p19 mutant-SARS CoV 2019+RNase III EEQ mutant generated inclusion body, the tenth lane is TBSV p19-SARS CoV 2019+RNase III Sweetpotato chlorotic stunt virus (SPCSV) mutant generated inclusion body, the 11^{th} lane is CIRV 15/16 p19 Mutant-SARS CoV 2019+RNase III SPCSV mutant generated inclusion body.

As shown in FIG. 3E, the 12^{th} lane is chemically synthesized siRNA, the 13^{th} lane is CIRV 15/16 p19 mutant-SARS CoV 2019+RNase III Ct produced dsRNA, the 14^{th} lane is CIRV 15/16 p19 mutant-SARS CoV 2019+RNase III Lm produced dsRNA, the 15^{th} lane is CIRV 15/16 p19 mutant-SARS CoV 2019+RNase III Mt produced dsRNA, the 16^{th} lane is CIRV 15/16 p19 mutant-SARS CoV 2019+RNase III Msp produced dsRNA, the 17^{th} lane is TBSV p19-SARS CoV 2019+RNase III E38A mutant produced dsRNA, the 18^{th} lane is CIRV 15/16 p19 mutant-SARS CoV 2019+RNase III E38A mutant produced dsRNA, the 19^{th} lane is TBSV p19-SARS CoV 2019+RNase III EEQ mutant produced dsRNA, the 20^{th} lane is CIRV 15/16 p19 mutant-SARS CoV 2019+RNase III EEQ mutant produced dsRNA, the 21^{st} lane is TBSV p19-SARS CoV 2019+RNase III SPCSV produced dsRNA, the 22^{nd} lane is CIRV 15/16 p19 mutant-SARS CoV 2019+RNase III SPCSV produced dsRNA.

As used herein, “substantially complementary” refers to a first nucleotide sequence having at least 90% complementarity over the entire length of the sequence with a second nucleotide sequence, e.g. 90% complementary, 95% complementary, 98% complementary, 99% complementary, or 100% complementary. Two nucleotide sequences can be substantially complementary even if less than 100% of the bases are complementary, e.g. the sequences can be mismatched at certain base.

As used herein, the term “target RNA” refers to an RNA present in a cell (i.e. the “target cell”). The target RNA comprises a target sequence to which one strand of a siRNA according to the methods and compositions described herein binds, thereby causing RNAi silencing of the target RNA. The target cell can be the bacterial cell comprising a siRNA-binding polypeptide or another cell, either prokaryotic or eukaryotic. The target sequence can be an RNA that can be translated (i.e. it can encode a polypeptide, e.g. mRNA) or it can be an RNA that is not translated (i.e. a non-coding RNA). In some embodiments, the target sequence can be any portion of an mRNA. In some embodiments, the target sequence can be a sequence endogenous to the cell. In some embodiments, the target sequence can be a sequence exogenous to the cell. In some embodiments, the target sequence can be sequence from an organism that is pathogenic to the target cell, e.g. the target sequence can be sequence from a viral, bacterial, fungal, and/or parasitic origin. In some embodiments, the target sequence is a viral nucleotide sequence.

In an embodiment, the siRNAs can be generated from the dsRNA comprising a nucleic acid sequence substantially complementary to a target RNA. As used herein, the term “siRNA” refers to a nucleic acid that forms an RNA molecule comprising two individual strands of RNA which are substantially complementary to each other. Typically, the siRNA is at least about 15-40 nucleotides in length (e.g., each complementary sequence of the double stranded siRNA is about 15-40 nucleotides in length, and the double stranded siRNA is about 15-40 base pairs in length, preferably about 19-25 base nucleotides, e.g., 19, 20, 21, 22, 23, 24, or 25 nucleotides in length). In some embodiments, a siRNA can be blunt-ended. In some embodiments, a siRNA can comprise a 3′ and/or 5′overhang on each strand having a length of about 0, 1, 2, 3, 4, or 5 nucleotides. The length of the overhang is independent between the two strands, i.e., the length of the overhang on one strand is not dependent on the length of the overhang on the second strand. The siRNA molecules can also comprise a 3′hydroxyl group. In some embodiments, the siRNA can comprise a 5′phosphate group. A siRNA has the ability to reduce or inhibit expression of a gene or target RNA when the siRNA is present or expressed in the same cell as the target gene, e.g. the target RNA. siRNA-dependent post-transcriptional silencing of gene expression involves cutting the target RNA molecule at a site guided by the siRNA.

Preferably, the vector further comprises a “P19-P1 fusion protein expression cassette” including a promoter and a sequence encoding a P19-P1 fusion as described below and optionally a siRNA-generating enzyme, in particular a ribonuclease like an Escherichia coli (E. coli) RNase III. One of skill in the art will understand that the method of the present invention could also use sequences encoding a siRNA-generating enzyme such as a RNase III from any other bacterial species. In preferred embodiments of the present invention, the vector is a plasmid further comprising a siRNA-binding polypeptide expression cassette including a promoter, a sequence encoding a siRNA-binding polypeptide and a sequence encoding a siRNA-generating enzyme, wherein the siRNA-binding polypeptide is a P19-P1 fusion protein and the siRNA-generating enzyme is an E. coli RNase III.

As show in FIG. 1, the one or more promoters in the vector preferably include a T7 promoter, i.e. a T7 promoter sequence which is known to one of skill in the art. In particular, the vector comprises two or more promoters, in particular two or more T7 promoters. The “siRNA producing cassette” in the cloned vector preferably comprises two opposing promoters, more preferably T7 promoters, with the at least one dsDNA fragment in between.

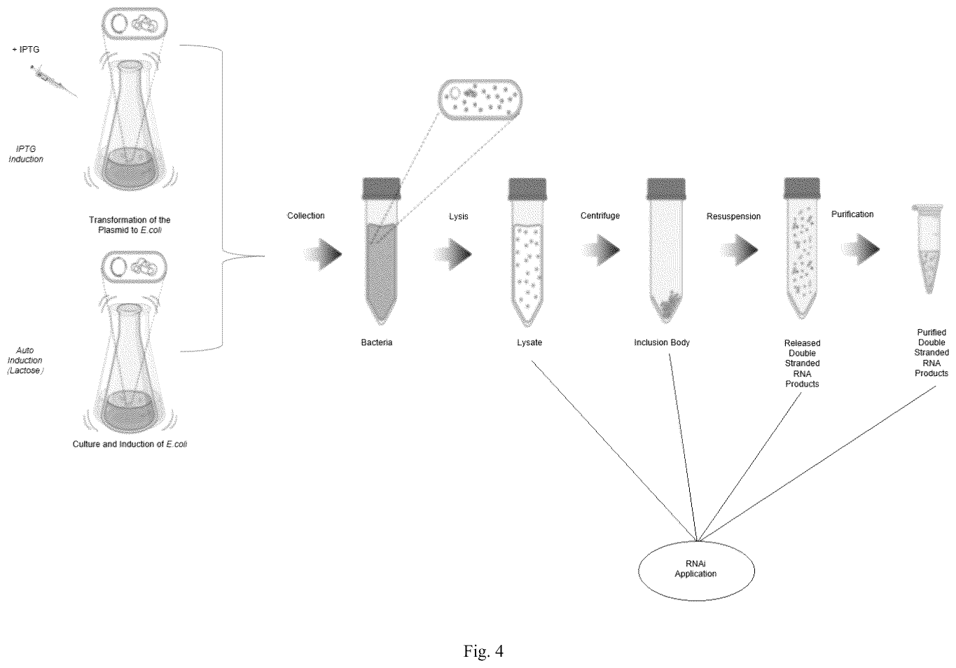

Referring to FIG. 4 illustrated the method for double-stranded RNA producing, the double-stranded RNA producing method includes follow steps.

Firstly, the vector used herein, such as plasmid, is transformed to bacteria such as Escherichia coli cell (E. coli). Alternatively, the bacterial cell can be from any species although E. coli cells are preferred.

Secondly, culturing and inducing the bacteria either through IPTG induction method or lactose auto-induction method to express the vector.

Thirdly, extracting the inclusion body by the lysis of the bacteria.

The term “extracting” as used herein means separating the siRNA from other components such as from the bacterial cells and other DNA or RNA sequences or polypeptides that are present resulting from the materials used and conditions applied for producing the double-stranded RNAs. In particular, isolating the double-stranded RNAs comprises extracting and purifying the double-stranded RNAs.

In some embodiments, the inclusion body can be resuspended and the released double-stranded RNAs may continually purify to generate the purified double-stranded RNAs.

In some embodiments, the bacteria cell lysates, released or purified ds RNAs and the inclusion body can also be used together for any suitable RNAi applications, according to the actual needs.

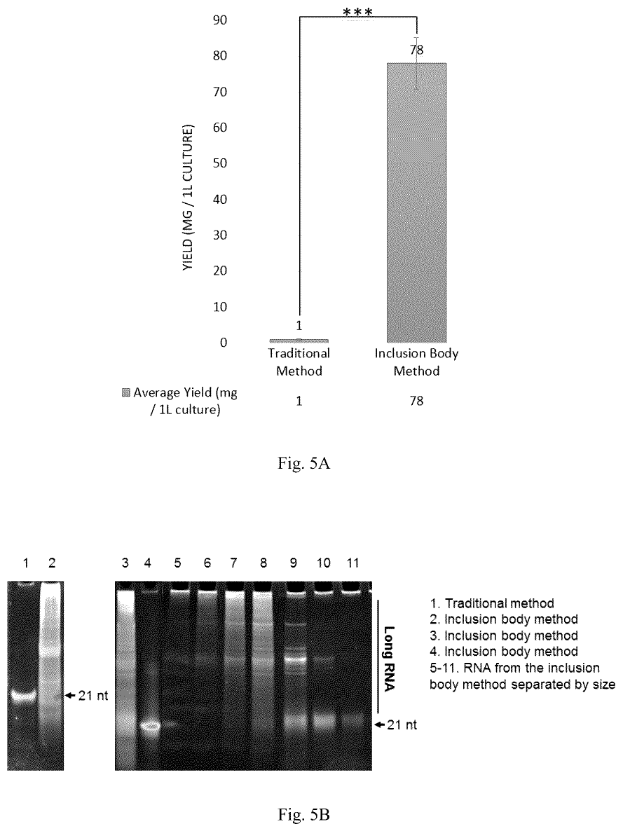

Referring to FIGS. 5A and 5B illustrated the yield comparison between traditional method and inclusion body method for producing double-stranded RNA. The inclusion body method for producing double-stranded RNA disclosed by the application significantly increases the yield of double-stranded RNA. As shown in FIG. 5B, lines 2-3 are the double-stranded RNA produced by the inclusion body method, lines 5-11 are the result of double-stranded RNA produced by inclusion body method separated by size.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed is:

1. A vector comprising:
a first nucleic acid sequence encoding a siRNA-binding polypeptide comprising p19 protein and protein 1; and
a second nucleic acid sequence substantially complementary to a target RNA.

2. The vector of claim 1, wherein the vector further comprises a third nucleic acid sequence encoding an siRNA-generating enzyme, and the siRNA-generating enzyme is selected from RNase III and/or RNase III mutant at least 95% identical to the RNase III, wherein the RNase III from C. trachomatis, L. monocytogenes, M. tuberculosis, Mycoplasma sp., Sweetpotato chlorotic stunt virus.

3. The vector of claim 1, wherein the p19 protein is selected from following proteins:
TBSV p19 protein;
TBSV p19 protein mutant at least 95% identical to the TBSV p19 protein;
CIRV p19 protein; or
CIRV p19 protein mutant at least 95% identical to the CIRV p19 protein.

4. The vector of claim 1, wherein the protein 1 is selected from following proteins:
SARS CoV 2019 (SARS-CoV-2);
SARS CoV 2003 (SARS-CoV-1);
Toxin (Bacillus thuringiensis-Cry1Ab/Cry1Ac/Cry1I); or
protein configured to generate inclusion body.

5. The vector of claim 4, wherein the nucleic acid sequence encodes for protein 1 as set forth in SEQ ID NO: 1, SEQ ID NO: 2, SEQ ID NO: 3, SEQ ID NO: 4 or SEQ ID NO:5.

6. The vector of claim 1, wherein the siRNA-binding polypeptide comprises a purification tag, or without the purification tag.

7. The vector of claim 1, wherein the vector comprises a promoter sequence.

8. The vector of claim 1, wherein the vector is a plasmid expressed by Escherichia coli cell, or other bacterial cells.

9. An inclusion body expressed by bacteria, wherein the inclusion body encodes by the vector described of claim 1.

10. An insoluble complex, generated by the interaction of a fusion protein of p19 and a double stranded RNAs of target sequence.

11. The insoluble complex of claim 10, wherein the fusion protein of p19 at least comprises p19 protein and protein 1.

12. A method for double-stranded RNA producing, comprising:
transforming a vector to bacteria;
culturing and inducing the bacteria expressed the inclusion body described of claim 9;
extracting the inclusion body by the sonication or high-pressure lysis of the collected bacteria;
wherein the vector comprises a first nucleic acid sequence encoding a siRNA-binding polypeptide comprising p19 protein and protein 1; and a second nucleic acid sequence substantially complementary to a target RNA.

13. The method of claim 12, wherein the bacterium is an Escherichia coli cell.

14. The method of claim 12, wherein the method further comprises: resuspending the inclusion body to produce a released double-stranded RNA purifying the released double-stranded RNA.

15. The method of claim 12, wherein the step of extracting the inclusion body further comprises:
crushing the collected bacteria by sonication or high-pressure lysis method;
extracting the inclusion body from the crushed bacteria by centrifugation.

16. A double-stranded RNA and/or inclusion body containing the double-stranded RNA produced by the method of claim 12.

17. A method of RNAi comprising application of the double-stranded RNA and/or inclusion body containing the double-stranded RNA of claim 16 to a subject in need of RNA interference, wherein the subject comprises eukaryotic cells and/or organisms.

18. An inclusion body for use in the purification of RNA product, wherein the inclusion body is formed by protein 1 fused with RNA-binding protein.
